(19) United States
(12) Patent Application Publication
(10) Pub. No.: US 2005/0287042 A1
(43) Pub. Date: Dec. 29, 2005

(54) Nanofibers with modified optical properties

(76) Inventors: George G. Chase, Wadsworth, OH (US); Edward A. Evans, Hudson, OH (US); Rex D. Ramsier, Berlin Center, OH (US); Darrel H. Reneker, Akron, OH (US); Daniel J. Smith, Stow, OH (US)

Correspondence Address: ROETZEL AND ANDRESS, 222 SOUTH MAIN STREET, AKRON, OH, 44308, US

(21) Appl. No.: 10/525,693

(22) PCT Filed: Aug. 22, 2003
(86) PCT No.: PCT/US03/26449

§ 371 Date: Jul. 18, 2005

Related U.S. Application Data
(60) Provisional application No. 60/405,129, filed on Aug. 22, 2002.

(51) Int. Cl. G01N 33/00

(52) U.S. Cl. 422/88

(57) ABSTRACT

Nanofibers modified to alter their optical properties in the infrared part of the electromagnetic spectrum, which nanofibers can be used in applications ranging from identification technology to energy conversion devices (e.g., thermophotovoltaics) to stealth technology. The desired optical properties can be obtained by modifying the fibers with rare earth and other materials and then can be incorporated into garments or other composite structures or can be applied as coatings on solid surfaces, to be used in a number of applications that benefit from selective emission properties.

### BACKGROUND OF THE INVENTION

This invention concerns the spectral and directional modification of the optical absorptivity, emissivity, and reflectivity of nanofibers and nanofiber-based structures, in specific regions of the electromagnetic spectrum as a function of temperature and chemical environment. This invention covers the production, modification, and application of nanofiber-based systems having controlled optical properties. More broadly, this invention focuses on providing unique selective emitter systems that are comprised of nanofibers, and applications for such selective emitters.

Selective emitters are generally known, and serve to convert thermal energy into narrow band radiation. Depending upon the type of selective emitter, this spectral output may broadly range across the electromagnetic spectrum. Commonly, the narrow band output of the selective emitter is either in the visible region of the electromagnetic spectrum, thereby often serving as a light source (e.g., lantern mantels), or in the infrared region of the spectrum, thereby being useful in energy conversion applications (e.g., thermophotovoltaics).

Selective emitter materials made from rare-earth and other metal oxides are available in the prior art and provide the proper spectral distributions for applications such as those broadly mentioned above. However, it is appreciated in the art that selective emitters, to date, have suffered from inefficiency, mechanical instability, and low thermal conductivity. This invention serves to advance the state of the art by employing nanofibers as selective emitters.

### SUMMARY OF THE INVENTION

It has been found that nanofibers can be modified to alter their optical properties in the infrared part of the electromagnetic spectrum. These modified nanofibers can be used in applications ranging from identification technology to energy conversion devices (e.g., thermophotovoltaics) to stealth technology. The desired optical properties can be obtained by modifying the fibers with rare earth and other materials. These optically modified nanofibers can then be incorporated into garments or other composite structures or can be applied as coatings on solid surfaces, to be used in a number of applications that benefit from selective emission properties.

Regarding identification technologies, modification of the optical emissivity of nanofibers by a large fraction in a narrow band of the infrared spectrum would render these nanofibers detectable only by specific viewing devices tailored to view the electromagnetic spectrum in that narrow band. Military applications might be envisioned, wherein clothing and other surfaces can be modified for signature reduction, while enabling insertion team self-identification. Invisible tagging of the clothing of personnel in potential hostage situations could aid rescue operations. Military subcontractor cites could be made more secure by placing these invisible markers in uniforms. This concept would also have a number of non-military applications as well.

With respect to energy conversion, it is envisioned that the nanofibers of this invention, when properly employed, would yield energy conversion systems significantly more efficient than those of the prior art. Because the volume of a nanofiber is essentially near its surface, the selective emitter systems of this invention are finely tunable, provide rapid and efficient heat transfer, and may provide opportunities for modifying their optical properties, due to the fact that their optical properties are strongly coupled with their surface chemistry. Additionally, the large aspect ratio of nanofibers increases the structural and mechanical stability of the selective emitter systems, improves the fluid dynamics surrounding the systems, and leads to speciously anisotropic and tunable optical response.

### DESCRIPTION OF THE DRAWINGS

FIG. 1 is a graph of the absorption spectra of PDPP nanofibers modified by erbium;

FIG. 2 is a graph of the absorption spectra of untreated, coated, and doped PDPP fibers with Er (III) nitrate;

FIG. 3 is a scanning electron microscope image of PDPP electrospun nanofibers coated with Er(III) nitrate;

FIG. 4 is a graph showing the temperature stability of both PDPP and SiO fibers;

FIG. 5 is a graph of the absorption spectra of untreated, coated, and doped SiO fibers with Er(III) nitrate;

FIG. 6 is a scanning electron microscope image of SiO electrospun nanofibers coated with Er(III) nitrate;

FIG. 7 is a scanning electron microscope image of SiO electrospun nanofibers after annealing to 800° C.; and

FIG. 8 is a graph of the emittance intensity of titania nanofibers modified by erbium.

### DESCRIPTION

In this invention, nanofibers are optically modified to respond to thermal energy to emit radiation within a narrow band of the electromagnetic spectrum. More particularly, nanofibers are coated or doped with optical materials that cause the nanofibers to exhibit the desired spectral output. The extremely small diameter of the electrospun nanofiber makes it essentially an isothermal surface, with very little volume and relatively large surface area The large surface area per unit mass will significantly increase its response to external stimuli such as electromagnetic fields and thermal energy transfer, thus increasing the efficiency of it optical output. Additionally, the large aspect ratio (length/diameter) inherent in nanofibers will provide improved mechanical stability by alleviating axial stresses and allowing for flexing in many applications wherein composite structures of these nanofibers are employed.

The nanofiber materials may be selected from virtually any material that is capable of forming nanofibers and further capable of being coated or doped with suitable optical materials. Without limitation, the nanofiber material may be selected from polymer nanofibers, carbon fiber nanofibers, and ceramic nanofibers. It is preferable that the nanofiber material be stable at high temperatures, such as, for example up to 1500° C., especially when the optically modified nanofiber end product is to be employed in energy conversion systems, such as thermophotovoltaic devices. The nanofibers may be employed as nanofibers per se, or as more composite woven or non-woven structures.

The optical materials of this invention that are employed to provide nanofibers with the desired spectral narrow band emission properties are generally known, and may include metals, metal oxides, rare earth metals, and group IV materials according to new IUPAC notation. The rare earth metals are particularly preferred and include, by way of non-limiting example, cerium, praseodymium, neodymium, samarium, europium, gadolinium, terbium, dysprosium, holmium, erbium, thulium, ytterbium, lutetium, their oxides, carbides, borides, and nitrides, and mixtures of the foregoing.

The nanofibers may be made by any technique known for producing fibers with cross sections of nanoscale dimension. Electrospinning is particularly preferred for nanofiber materials capable of forming nanofibers through such a process. And it's well known that typical electrospinning processes can produce single nanofibers that are often collected onto a mandrel. The nanofibers may be coated with the optical materials through known techniques such as sol gel and vapor phase deposition. According to this invention, the nanofibers may also be doped with these optical materials, wherein it should be understood that by [symbol] doping [symbol] it is meant that the optical material is incorporated into the nanofiber, as opposed to being a surface coating, through either chemical or physical interaction between the fiber material and the optical material. Optical [symbol] coating”, thus, is not to be understood as being limited to surface coating and could include partial coatings or coatings in which a portion of the coating is imbedded in the surface. Further, doped nanofibers can be manufactured by incorporating the optical material into the electrospinable solution, and the resulting nanofiber has optical materials that are embedded or tethered into the nanofiber.

The amount of the optical material will be a sufficient or effective amount such that the doped or coated nanofibers will produce a response to thermal energy and emit detectable radiation. Generally the amount will be in the range of from about 5% by weight based upon the weight of the nanofibers. For the coated nanofibers, the preferred amount is about 10% to 45% by weight, with 15% to 45% by weight being also preferred. For the doped nanofibers, the preferred amount is about 10% to 35% by weight, with 15% to 30% by weight being also preferred.

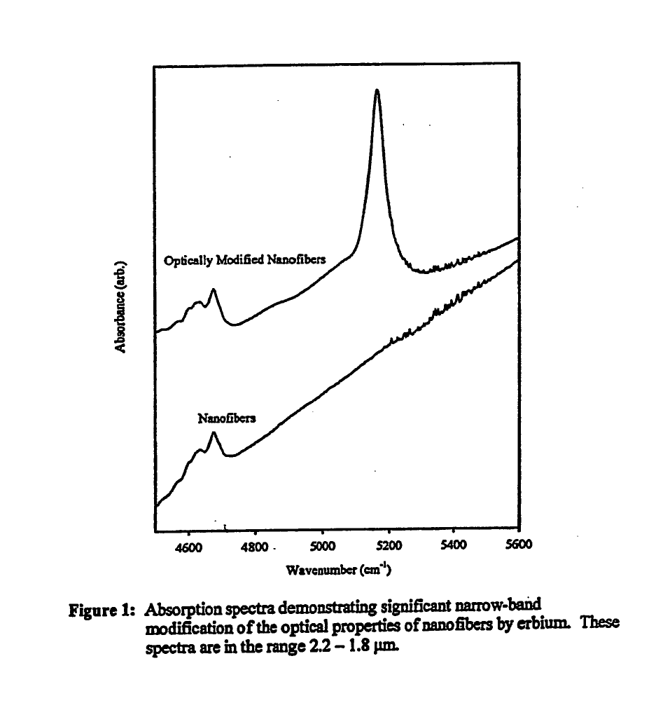

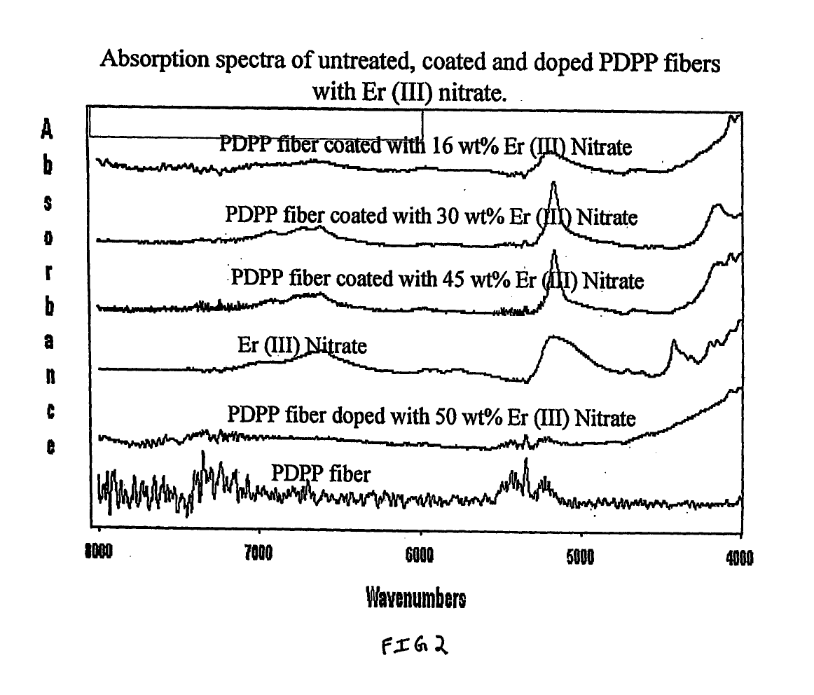

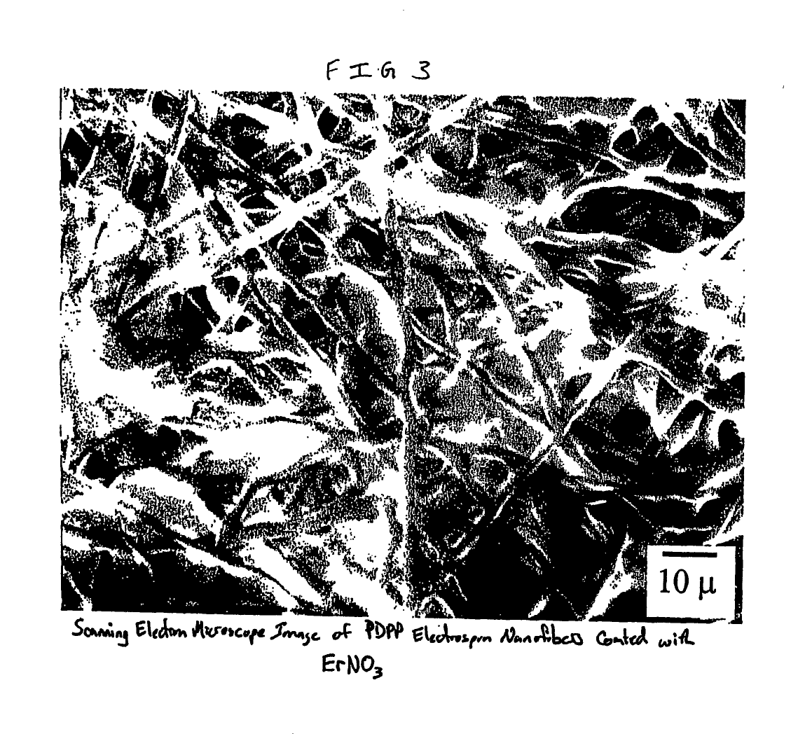

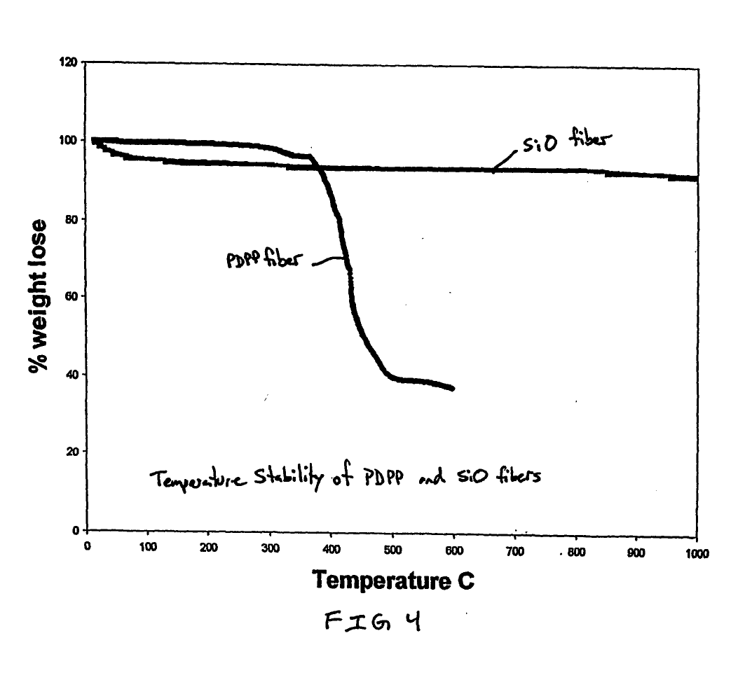

FIG. 1 shows the absorption spectra of polydiphenoxyphosphazene (PDPP) nanofibers coated with erbium (Er) from Er (III) nitrate hydrate dissolved in ethanol. The Fig. exhibits that nanofibers may be made out of precursors for PDPP, that these nanofibers can be coated with erbium, and that these coatings can be used to selectively modify the optical properties of nanofibers in the infrared region of the electromagnetic spectrum. As seen in the Fig., infrared absorption spectroscopy indicates the spectral modification of the high temperature PDPP nanofibers by erbium. These coated nanofibers have been annealed to temperatures greater than 200° F., for up to one hour, in air, and no degradation of the PDPP cores or Er-based coatings has been detected. In FIG. 2, the absorption spectra of PDPP fibers coated with Er (III) nitrate, in differing amounts (16 wt %, 30 wt %, and 45 wt %), and PDPP fibers doped with 50 wt % Er(III) nitrate are compared with the absorption spectra of an uncoated, undoped PDPP fiber and the absorption spectra of Er (III) nitrate. The coated PDPP fibers showed significant absorbance in the near IR, while doping of the PDPP fibers did not produce significant absorbance in the near IR. FIG. 3 shows a scanning electron microscope image of PDPP electrospun nanofibers coated with Er (III) nitrate. In FIG. 4, it can be seen that the PDPP fiber (uncoated, undoped) showed temperature stability until approximately 370° C.

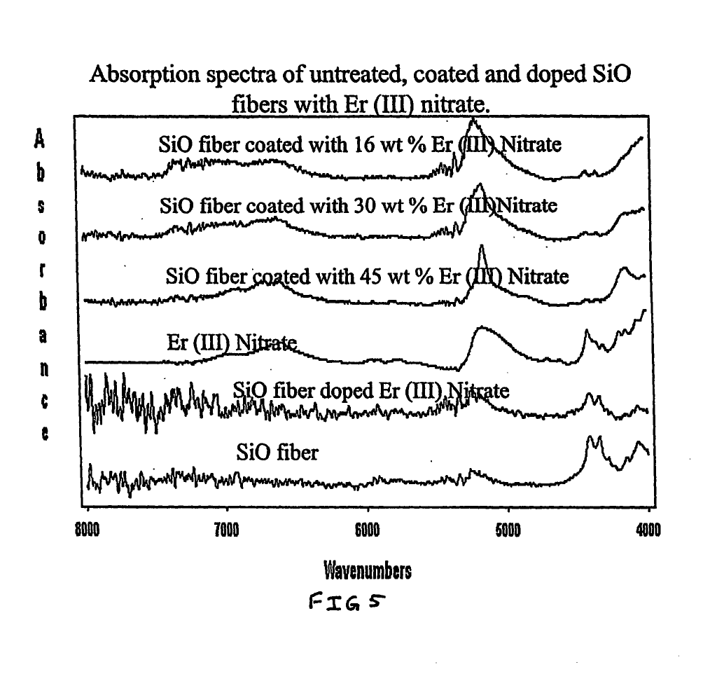

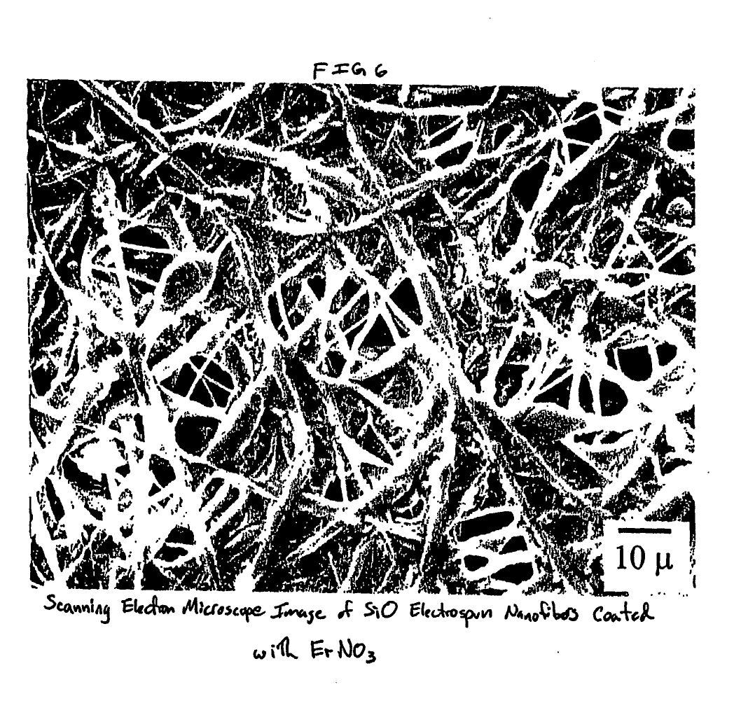

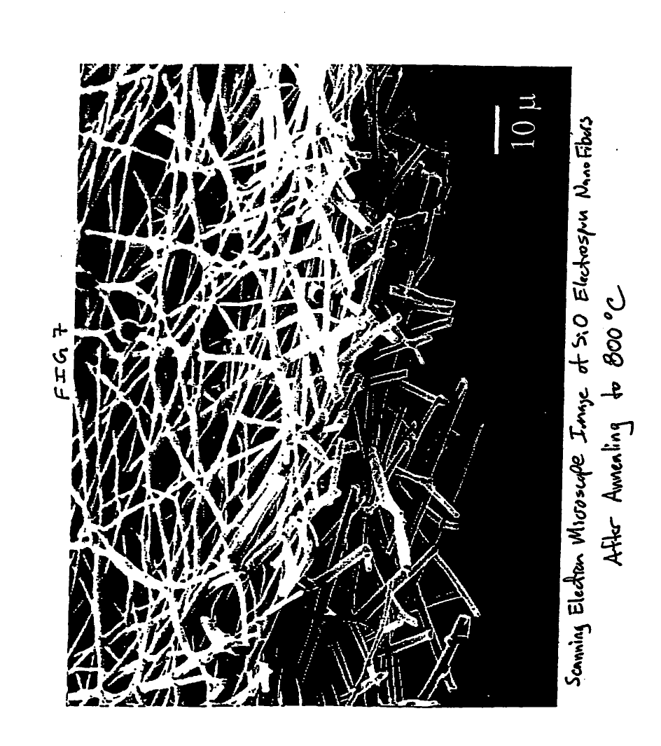

FIG. 5 shows the absorptions spectra of SiO nanofibers coated with erbium (Er) from Er (III) nitrate hydrate dissolved in ethanol. The Fig. exhibits that nanofibers may be made out of precursors for SiO, that these nanofibers can be coated with erbium, and that these coatings can be used to selectively modify the optical properties of nanofibers in the infrared region of the electromagnetic spectrum. As seen in FIG. 5, infrared absorption spectroscopy indicates the spectral modification of the high temperature SiO nanofibers by erbium. In FIG. 5, the absorption spectra of SiO fibers coated with Er (III) nitrate, in differing amounts (16 wt %, 30 wt %, and 45 wt %), and SiO fibers doped with 50 wt % Er(III) nitrate are compared with the absorption spectra of an uncoated, undoped SiO fiber and the absorption spectra of Er (III) nitrate. The coated SiO fibers showed significant absorbance in the near IR, while doping of the SiO fibers did not produce significant absorbance in the near IR FIG. 6 shows a scanning electron microscope image of SiO electrospun nanofibers coated with Er (III) nitrate. In FIG. 4, it can be seen that the SiO fiber (uncoated, undoped) showed temperature stability until approximately 370° C. In FIG. 7, it can be seen that the SiO electrospun nanofibers are stable after annealing to 800° C.

Recalling that nanofibers are too small to be seen by the human eye, and can be woven into garments leaving the clothing visually and functionally unchanged, it should be appreciated that these optically modified nanofibers can be employed for remote identification purposes. Clothing or cloth patches which are attached to clothing and/or other surfaces can essentially be invisibly tagged with these optically modified nanofibers, such that, although they appear common to the human eye, they would appear to be lit up when viewed through a viewing device that is tailored to view the particular spectrum output of the nanofibers.

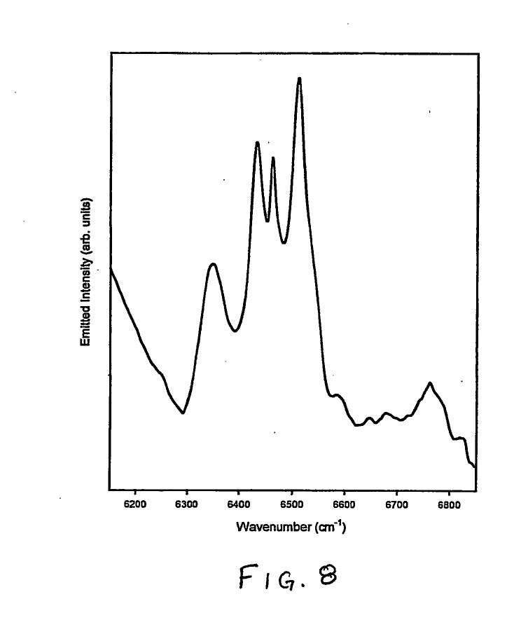

These nanofibers might also be employed in energy conversions systems, namely, thermophotovoltaic (TPV) devices. The increased power density afforded by the nanofibers implies that the operational temperature differential can be lowered. This, in turn, means that electrical power generation might be achieved from lower temperature sources, such as the waste heat rejected from vehicles, and perhaps even the human body. Waste heat from a vehicle could be converted to electricity, which in turn could be used for a number of beneficial purposes. By combining photovoltaic cells with the nanofibers selective emitters woven into clothing, it might be possible to provide a source of electricity from a person [symbol] s body heat. If the waste heat from objects such as the human body and vehicles could be converted to electricity in this manner, it would make the vehicles and bodies less susceptible to detection by thermal imaging devices. For example, as shown in the graph in FIG. 8, a self-supporting titania nanofiber mat, doped with erbia will emit in the near-IR when heated by hot gas convection from a propane flame.

It is also envisioned that the coated or doped nanofibers of this invention could be coated or impregnated with catalyst particles that could be selected to produce heat through exothermic reactions with reagents exposed to the nanofibers. This heat, produced locally on the catalyst particles, would be effectively transferred to adjacent nanofibers according to the invention, which would radiate light in a specific narrow region of the electromagnetic spectrum. The light would then be converted to useful energy through photovoltaic cells. It should also be appreciated that these catalyst/nanofiber composites could be employed as chemical or biological agent sensors. In such an application, the catalyst/nanofiber composite would change optical properties when a target agent reached the catalyst, which would be selected to exothermally react with that agent.

Another application of the present invention would involve the doping and/or coating of nanofibers with the various rare earth metals. This would allow for additive color mixing producing “colors” in near-IR portion of the spectrum. As is well known in visible coloration, a range of colors can be derived from three primary colors, namely red, green, and blue. Similarly, with the present invention, color mixing in the near-IR, can be done with Er, Ho, and Yb which can be employed as “red”, “green”, and “blue”, respectively. By adjusting the relative ratios (or tristimulus values) of these, the “color” of the modified nanofibers can be adjusted. Use of other rare earth metals will produce “color” with the near-IR spectra, and can be used per se or in combination with the “primary” colors to modify or adjust the “color” produced. For example, use of the fibers would allow for a method of tagging heated gas exhaust pipes such as vehicles' industrial exhaust and the like, and the “color” could be ˆ and monitored. Further, the nature of the nanofibers, i.e., high surface area, low volume, means that the exhaust system would not suffer from significant pressure drops.

Further, since the coatings applied have controllable roughness and morphology, there is a controllable spatial frequency. Combining controllable diameters, which is another spatial frequency, and color, can produce a continuously variable 3D-space for encoding information, which can be extracted via spectroscopy and Fourier analysis. This could make decoding of the information by another party difficult or impossible to achieve. Still further, aligning the nanofibers would allow for spacial and/or directional control of the emitted light.

In light of the foregoing, it should thus be evident that this invention, providing nanofibers with modified optical properties, substantially improves the art. While, in accordance with the patent statutes, only the preferred embodiments of the present invention have been described in detail hereinabove, the present invention is not to be limited thereto or thereby.

The foregoing embodiments of the present invention have been presented for the purposes of illustration and description. These descriptions and embodiments are not intended to be exhaustive or to limit the invention to the precise form disclosed, and obviously many modifications and variations are possible in light of the above disclosure. The embodiments were chosen and described in order to best explain the principle of the invention and its practical applications to thereby enable others skilled in the art to best utilize the invention in its various embodiments and with various modifications as are suited to the particular use contemplated. It is intended that the invention be defined by the following claims.

1. A nanofiber coated or doped with at least one optical material.

2. The nanofiber of claim 1 wherein the nanofiber is selected from the group consisting of a polymer nanofiber, a carbon fiber nanofiber, a ceramic nanofiber and mixtures thereof.

3. The nanofiber of claim 1 wherein the optical material is selected from the group consisting of metal, metal oxide, rare earth metal, group IV material, and mixtures thereof.

4. The nanofiber of claim 1 wherein the optical material is selected from the group consisting of cerium, praseodymium, neodymium, samarium, europium, gadolinium, terbium, dysprosium, holmium, erbium, thulium, ytterbium, lutetium, their oxides, carbides, borides, and nitrides, and mixtures thereof.

5. The nanofiber of claim 1 wherein the nanofiber is selected from the group consisting of polydiphenoxyphosphazene, SiO, titania, and mixtures thereof and the coating is selected from the group consisting of erbium, homia, ytterbia, and mixtures thereof.

6. The nanofiber of claim 1 wherein the nanofiber is additionally coated or impregnated with catalyst particles whereby the catalyst will produce heat through exothermic reactions with reagents exposed to the nanofibers.

7. The nanofiber of claim 1 wherein the nanofiber is additionally doped with rare earth metal or metals that can produce colors in the near-IR portion of the spectrum.

8. The nanofiber of claim 1 wherein the optical material is present in an effective amount to produce a response to thermal energy and to result in the emittance of detectable radiation.

9. The nanofiber of claim 1 wherein the optical material is present in an amount of 5% to 50% by weight based upon the weight of the nanofiber.

10. The nanofiber of claim 1 wherein the optical material is present in an amount of 10% to 45% by weight based upon the weight of the nanofiber.

11. The nanofiber of claim 1 wherein the optical material is present in an amount of 15% to 45% by weight based upon the weight of the nanofiber.

12. The nanofiber of claim 1 wherein the optical material is present in an amount of 10% to 35% by weight based upon the weight of the nanofiber.

13. The nanofiber of claim 1 wherein the optical material is present in an amount of 15% to 30% by weight based upon the weight of the nanofiber.

14. The nanofiber of claim 1 wherein the optical material is present in an amount of 5% to 50% by weight based upon the weight of the nanofiber.

15. The nanofiber of claim 1 wherein the nanofiber is additionally doped with rare earth metal or metals selected from the group consisting of erbia, homia, ytterbia, and mixtures thereof that can produce colors in the near-IR portion of the spectrum.

16. The use of the nanofiber of claim 6 as chemical or biological agent sensors.

17. The use of the nanofibers of claim 1 in energy conversion systems.

18. The use of the nanofiber of claim 1 in a thermophotovoltaic device.

19. Fabric that has incorporated the nanofiber of claim 1.

20. The use of the nanofiber of claim 1 to produce detectable near-IR radiation.
